(19) United States
(12) Patent Application Publication
(10) Pub. No.: US 2005/0239114 A1
(43) Pub. Date: Oct. 27, 2005

(54) Methods and devices for microarray image analysis

(75) Inventors: Jekwan Ryu, Cupertino, CA (US); Richard P. Rava, Redwood City, CA (US); Stephen P.A. Fodor, Palo Alto, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/101,043

(22) Filed: Apr. 6, 2005

Related U.S. Application Data
(63) Continuation-in-part of application No. 11/026,615, filed on Dec. 30, 2004.
(60) Provisional application No. 60/559,806, filed on Apr. 6, 2004.
(60) Provisional application No. 60/565,041, filed on Apr. 23, 2004.

(51) Int. Cl. C12Q 1/68; G06F 19/00; G01N 33/48; G01N 33/50

(52) U.S. Cl. 435/6; 702/20

(57) ABSTRACT

The present invention provides methods and devices for high sensitivity and high speed microarray optical imaging. The methods include using patterned excitation to obtain a series of images and analyzing the images to resolve probe intensities which reflect the hybridization or binding between target and probes. Probe feature information and patterned excitation (structured illumination) information are incorporated into the analysis.

This application is a continuation-in-part of U.S. patent application Ser. No. 11/026,615, filed on Dec. 30, 2004, which claims priority to U.S. Provisional Application No. 60/559,806, filed on Apr. 6, 2004; and U.S. Provisional Application No. 60/565,041, filed on Apr. 23, 2004. The '806, '041 and '615 applications are incorporated herein by reference in their entirety for all purposes.

### BACKGROUND OF THE INVENTION

This application is related to microarray image detection and analysis.

High density microarray technology has revolutionized biological analyses. It has been extensively used for clinical diagnostics, toxicology, genomics, drug discovery, environmental monitoring, genotyping and many other fields (Fodor, S. P.; Read, J. L.; Pirrung, M. C.; Stryer, L.; Lu, A. T.; Solas, D. Light-directed, spatially addressable parallel chemical synthesis, Science 251(4995), 767-73, 1991; Fodor, S. P.; Rava, R. P.; Huang, X. C.; Pease, A. C.; Holmes, C. P.; Adams, C. L., Multiplexed biochemical assays with biological chips, Nature 364(6437), 555-6, 1993; Pease, A. C.; Solas, D.; Sullivan, E. J.; Cronin, M. T.; Holmes, C. P.; Fodor, S. P., Light-generated oligonucleotide arrays for rapid DNA sequence analysis, Proceedings of the National Academy of Sciences of the United States of America 91(11), 5022-6, 1994). Fluorescence labels are frequently used for microarray detection. A variety of image acquisition devices, such as CCD (charge coupled device), are used for detecting binding patterns.

### SUMMARY OF THE INVENTION

In one aspect of the invention, a method for microarray detection is provided. In one aspect of the invention, methods and devices are provided for microarray detection using a series of structured, textured, or patterned excitation (referred herein as patterned excitation) images to achieve subpixel resolution in detecting probe intensities. The microarray can be a nucleic acid probe array such as a spotted array (e.g., with cDNA or short oligonucleotide probes), high density in situ synthesized arrays (such as the GeneChip® high density probe arrays manufactured by Affymetrix, Inc., Santa Clara, Calif.). The microarrays can also be protein or peptide arrays. Typically, the density of the microarrays is higher than 500, 5000, 50000, or 500,000 different probes per cm². The feature size of the probes (synthesis area or immobilization area) is typically smaller than 500, 150, 25, 9, 5, 3 or 1 μm².

In one aspect of the invention, a method for microarray analysis is provided. The method includes obtaining a series of fluorescent images of a microarray, where the fluorescent signals reflect binding between targets and probes, and where each of the images is obtained with a different excitation pattern; and analyzing the images using calibrated information about the different excitation patterns and probe feature information to obtain intensities for each of the probes. The method typically includes generating a composite image where the composite image has a higher resolution than those of the fluorescent images. The different excitation patterns are generated by translating excitation patterns and different laser beam pair configurations.

One of skill in the art would appreciate that many different methods may be used to generate light patterns that can be used with patterned excitation. The methods of the invention are not limited to any particular methods for generating light patterns.

Typically, the images are obtained using a photo detector array. However, a single photo detector, such as a PMT may be used in some embodiments. The photo detector array can be a charge coupled device (CCD) such as an electron multiplication CCD (EMCCD). CMOS imagers such as an Active Pixel Sensor (APS) may also be used.

Information about different excitation patterns may include spatial frequency information such as orientation and spacing between adjacent peak intensities. In some embodiments, the analyzing steps include extracting cosine parameters to obtain I_{DC}, (DC component of intensity values), I_{AC} (AC component of intensity values), and φ (timing information, where the peak intensity appears) of pixel intensities. In a preferred embodiment, the analyzing step includes constructing a system of linear equations that relate the pixel intensities, subpixel weighting functions, and unknown subpixel intensities. For example, the linear equations may be as follows:

$${b}^{i} ( k ) = {\sum }_{m} {\sum }_{n} {W}^{i} ( m . , n , k ) {I}^{i} ( m , n ) ,$$

where Iⁱ(m,n) is the unknown subpixel intensities; Wⁱ(m,n, k) is the weighting function within i-th pixel for k-th frame at a subpixel location (m,n); and bⁱ(k) is the sequence of gray intensity values of i-th pixel. The equations may be solved to obtain subpixel intensities.

The weighting function Wⁱ(m,n, k) can be calculated, for example, using pattern calibration parameters as: E_{DC}+E_{AC}·cos(kₓ·x+k_{y}·y+φ), where E_{DC} and E_{AC} are DC and AC components of the pattern intensities, respectively; kₓ and k_{y} are x and y components of the pattern spatial frequency, respectfully; and the φ represents subpixel position of the pattern. Alternatively, the weighting function Wⁱ(m,n, k) is calculated by solving the equation

$${b}^{i} ( k ) = {\sum }_{m} {\sum }_{n} {W}^{i} ( m . , n , k ) {I}^{i} ( m , n )$$

using data obtained with reference samples with known subpixel intensities.

In another aspect of the invention, the intensity values are estimated using optimization methods. In some embodiments, the subpixel intensities are estimated with probe feature information as constraints. For example, the regularity of the probe features is used as constraints. The dynamic range the probe intensities can also be used. One particularly preferred method is to minimize

$${‖ {b}^{i} ( k ) - {\sum }_{m} {\sum }_{n} {W}^{i} ( m , n , k ) {I}^{i} ( m , n ) ‖}^{2} .$$

Liner programming is a preferred method for estimating the intensity values.

In another aspect of the invention, computer software products for microarray analysis are provided. Such products typically have a computer readable medium containing computer-executable instructions for performing the method of the invention. The software code structures typically include components for executing various steps of the methods.

In yet another aspect of the invention, a system for performing the methods of the invention is provided. Such a system typically includes a computer processor; and a memory coupled with the processor, the memory storing a plurality of machine instructions that cause the processor to perform logical steps of the methods of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1. Exemplary Experimental setup that generates patterned excitation using the interference of two coherent beams. A single source beam (model 177-G11-FBR, Spectra-Physics, Mountain View, Calif.) with 488 nm wavelength was split into two beams using a non-polarizing beam splitter (BS). The source beam was a linearly polarized with 0.7 mm beam diameter. Each of the two beams from the beam splitter was steered by two mirrors (M1-M3 or M2-M4) until they overlap and produce patterned excitation onto the sample region on the stage. Fluoresced photons from the sample are collected and recorded using a CCD imaging setup described in FIG. 2. The beams travel horizontally relative to the table until they reflected off the two final mirrors; M1 and M2. These two mirrors are tilted such that the beams travel downward after the reflection. For both beams, the angle between a line perpendicular to the horizontal surface and the beam was roughly 75 degrees. One of the steering mirrors, M4, was glued to a piezo-electric transducer (PZT, model P-841.10, Physik Instrument, Tustin, Calif.). By controlling the position of the mirror using PZT, the optical phase of one of the two beams can be controlled.

FIG. 2. Exemplary CCD imaging setup. Upon excited by the patterned excitation described in FIG. 1, a sample emits fluorescent photons that are collected and recorded by a CCD imaging setup shown in this photograph. A standard Affymetrix cartridge used as a sample is shown on top of a stage. The fluoresced photons from the sample are collected by a microscope objective (model CFI Plan Fluor 10×, Nikon Instruments Inc., Melville, N.Y.) and then pass through a long-pass filter (model CG-OG-515-1.00-3, CVI Laser Corporation, Livermore, Calif.) and a tube lens (model NT56-125, Edmund Industrial Optics, Barrington, N.J.) before they are projected onto a CCD camera (model DV887-FI, Andor Technology, South Windsor, Conn.).

FIG. 3. Excitation using a single laser beam. The figure shows a single laser beam used as an excitation source. The angle θ defined in the figure is typically 75 degrees in the experiment.

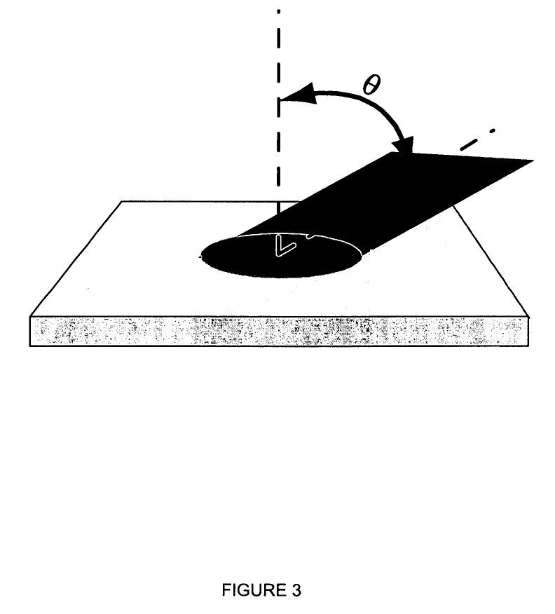

FIG. 4. CCD image of an Affymetrix standard microarray with 18 μm probe spacing excited by a single laser beam. A single laser beam (FIG. 3) with 6 mW optical power and 0.7 mm beam diameter was used as an excitation source and the CCD imaging setup in FIG. 2 was used to record an image of a standard Affymetrix array. The angle between the beam and a line perpendicular to the horizontal surface (the angle θ in FIG. 3) was 75 degrees. This results in an estimated optical power of 1.6 mW/mm² on the top surface of the fused silica scan window. In this image acquisition, the gain of the CCD camera was turned on to enhance the detection of the low intensity probes, while saturating the high and mid intensity probes. The image shown is the average of 30 repeated acquisitions, each with 520 msec exposure time.

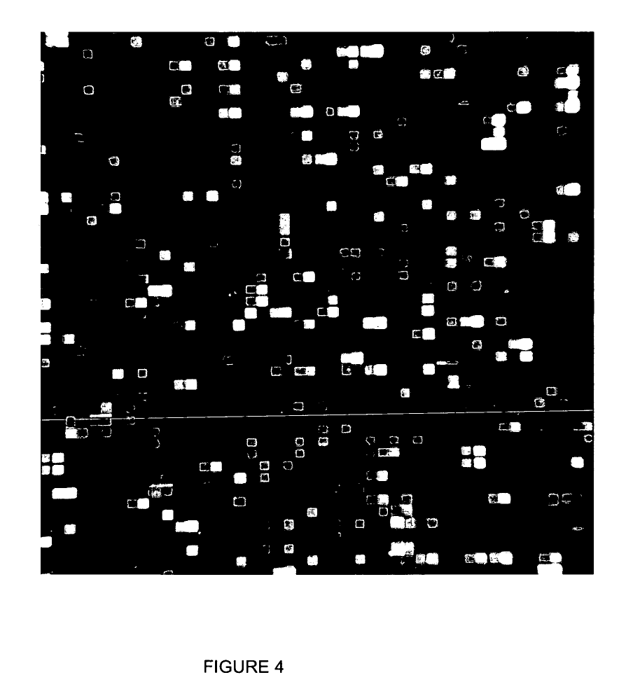

FIG. 5. Direct imaging of the interference pattern. To directly verify the generation of the high resolution optical pattern formed by the interference of two laser beams, a high power microscope objective with 100× magnification (model NT38-344, Edmund Industrial Optics, Barrington, N.J.) was used. The objective was positioned such that the focal plane of the objective lies in the region where the two beams overlap (bright spot in the photograph).

FIG. 6. Magnified image of the produced interference pattern projected onto the wall of the laboratory. Interference pattern made by a pair of laser beams is a sinusoidal brightness grating, also known as fringe pattern. The distance between adjacent two peak (or valley) intensities (the distance D defined in the figure) corresponds to the pitch or feature size of the interference pattern. Combined with the direction of the fringe pattern, this feature size of the interference pattern defines the spatial frequency of the pattern, that is, the vector k in the figure, in units of μm⁻¹. As will be explained in the following figure, the vector k is determined by the directions and the wavelength of the beams.

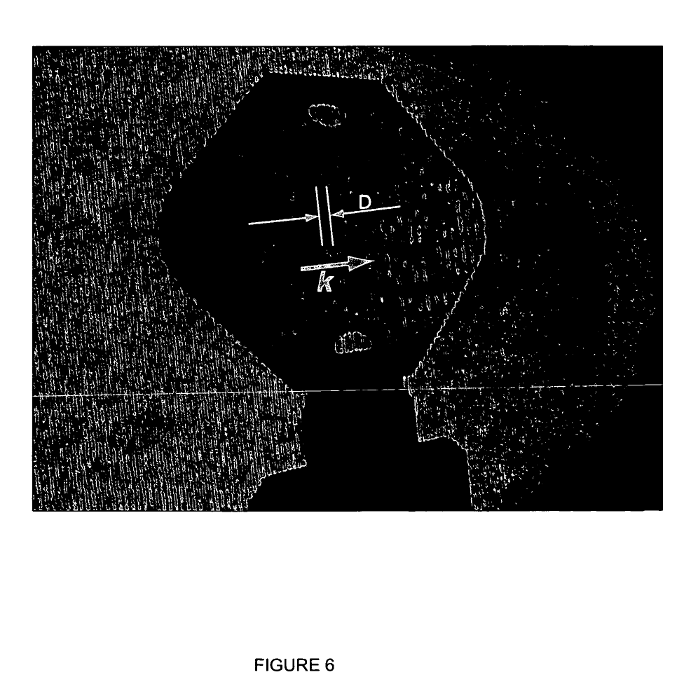

FIG. 7. Directions of the beams determines the spatial frequency of the projected interference pattern. The figure above shows the propagation vectors, or direction vectors, of the two interfering beams, indicated as vectors k₁ and k₂, respectively. The spatial frequency of resulting interference pattern is equivalent to the difference between these two vectors, that is, k₁−k₂.

FIG. 8. Directions of the two interfering beams can be conveniently defined in terms of angle. The separation angle φ on the figure left defines an angle between the two laser beams looking from the top. The half cone angle θ on the right figure corresponds to an angle between the laser beam and a line perpendicular to the horizontal surface. The angle θ is identical for both beams.

FIG. 9. Generating patterned excitation in the probe region of Affymetrix microarray. Unlike the situation where the two beams interfere in free air space, the beams undergo multiple refractions (and reflections as well) as they pass through several heterogenous regions. In case of the standard Affymetrix microarray, the beam from the air with refractive index n₁=1.0 pass through the 700 um thick fused silica layer with refractive index n₂=1.5 (for 488 nm wavelength), and finally reaches the probe region with refractive index n₃=1.3. The half cone angles defined in FIG. 8 at each different layer are indicated in the figure. When the cone angle in the air (1) is 75 degrees, the resulting cone angle in the probe region (θ3) becomes 48 degrees. The figure also shows a 100 nm diameter fluorescent sphere bonded to the bottom surface of the fused silica substrate. The size of this sphere was chosen to be a fraction of the wavelength of light such that the particle can spatially sample the resulting interference pattern. When the interference pattern is translated relative to the sphere, the brightness of the sphere will change depending on the position of the interference pattern. A photo detector that is placed on top can detect and record such brightness change.

FIG. 10. The brightness of the pixel that contains the calibration sphere encodes the sub-pixel position of the interference pattern. The box represents an area corresponding to a single pixel of the CCD camera that has the size of 1.6 μm in the image plane and the circle in the figure left represents a 100 nm diameter fluorescent sphere located inside the pixel. The figure on the left also shows a particular interference pattern overlaid on top of the sphere. The figure on the right shows the gray value of the same pixel on the left, indicating the brightness of the sphere illuminated by this particular excitation pattern on the left. If the interference pattern is translated relative to the fixed sphere, this will result in the systematic change in the brightness of the sphere, which is demonstrated experimentally in the next figure.

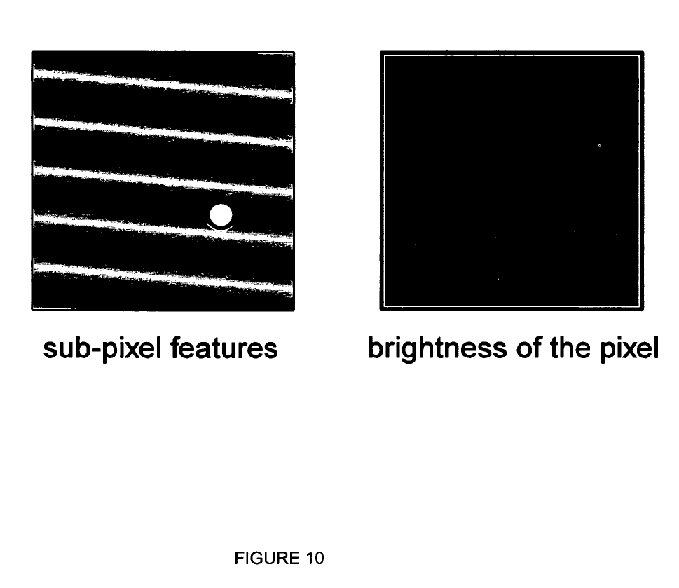

FIG. 11. Sinusoidal modulation of the brightness of a pixel containing the calibration particle demonstrates generation and control of the patterned excitation. A series of 30 patterned excitations were projected onto the sample in FIG. 9, by translating the same interference pattern using the piezo-electric transducer (PZT) shown in FIG. 1. Each time PZT moved to a new position, an image of the sample was acquired using the CCD camera with 1.2 sec exposure time. The top images show a series of nine images of an isolated 100 nm diameter fluorescent sphere. The plot at the bottom shows the brightness of the sphere measured by the gray value of the pixel that contains the sphere, as a function of the PZT motion. The result clearly shows a sinusoidal change in the brightness of the sphere as the excitation pattern is translated, demonstrating the generation and control of such patterned excitation pattern.

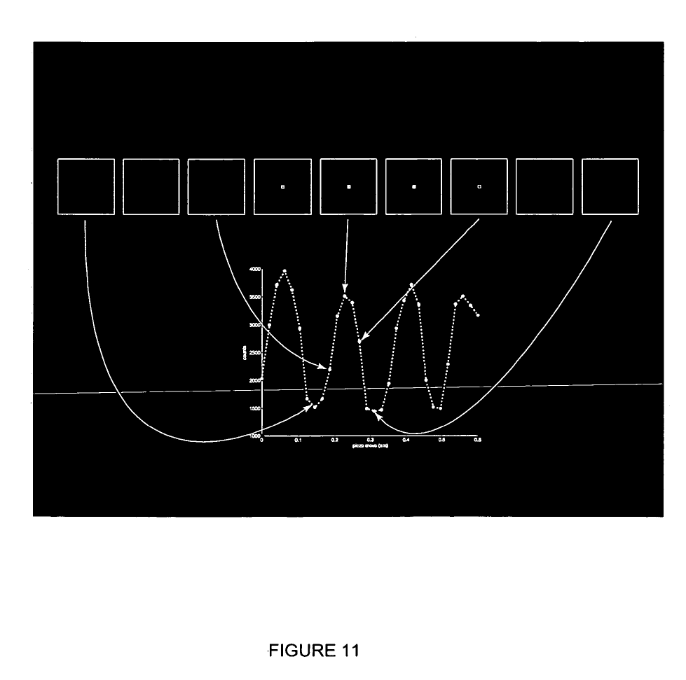

FIG. 12 shows a high level view of the imaging data structure for a software product for imaging analysis.

FIG. 13 shows subpixels with a detector pixel. The large squares represent the physical pixel of a detector. The small squares within a large square represent subpixels whose values are going to be determined through post processing. The raw images only contain intensity values for the pixels, not the subpixels. The following mathematical symbols can be used to illustrate an algorithm for constructing images with resolution higher than that of an image detector: i is the pixel number index, i=1, . . . , L; Iⁱ(m,n) is subpixel intensity of i-th pixel at a subpixel location (m,n); Wⁱ(m,n, k) is the weighting function within i-th pixel for k-th frame at a subpixel location (m,n), and finally, bⁱ(k) is the sequence of gray intensity values of i-th pixel.

FIG. 14 shows one exemplary embodiment of the algorithm for constructing an image. The series of intensities may be represented by bⁱ(k); weighting functions would be Wⁱ(m,n, k); and the equations can be

$${b}^{i} ( k ) = {\sum }_{m} {\sum }_{n} {W}^{i} ( m . , n , k ) {I}^{i} ( m , n ) ,$$

where Iⁱ(m,n) is the unknown subpixel intensities.

FIG. 15 is a system block diagram illustrating an exemplary embodiment of the pattern excitation microarray detection system.

FIG. 16 is a high level diagram illustrating the exemplary components for patterned excitation microarray detection of the invention.

FIG. 17 demonstrates spatially resolving subpixel probe intensities. The figure on the left is a conventional CCD image of a small region of a microarray with 5 μm probe spacing imaged using 6.4 μm image pixel size under conventional CCD imaging conditions; the image at the middle is a computer reconstructed image of the same region of the array acquired using the same lens and CCD with pattern excitation. Comparing the two images demonstrates the improvement in resolution, resolving subpixel intensities with the patterned excitation. The image on the right was acquired using 1.6 μm pixel size under conventional CCD imaging condition, showing a good correspondence with the patterned excitation image acquired at 6.4 μm pixel.

FIG. 18 shows a large area for the images at FIG. 17.

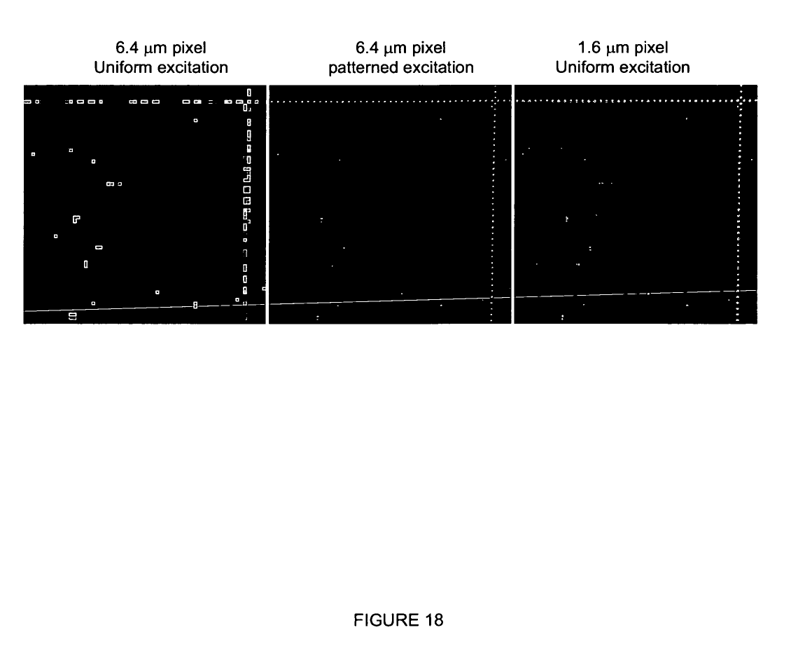

### DETAILED DESCRIPTION OF THE INVENTION

In one aspect of the invention, methods and devices are provided for microarray detection using a series of structured, textured, or patterned excitation (referred herein as patterned excitation) images to achieve subpixel resolution in detecting probe intensities. As used herein, subpixel refers to an area of a target region that is smaller than that of a single pixel in a detector. For example, in a 6.4 μm CCD detector, the size of one pixel of the CCD detector is about 6.4 μm×6.4 μm. Subpixel detection means detecting intensities of probes whose feature size is smaller than that the dimension of the detector pixel.

In such detections, probe feature information (the periodic geometry configuration of probes on a microarrary) can be incorporated into the analysis as constraints. In some embodiments, electron multiplying CCDs are used for imaging fluorescence emission patterns which indicate hybridization between probes and targets. However, as one of skill in the art would appreciate, this invention is not limited to any particular detection devices. Photo detection arrays (e.g., CCD, APS) are generally preferred. However, single detector, such as a PMT tube, could also be used.

I. General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. No. 60/319,253, Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent Application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

The present invention may also make use of the several embodiments of the array or arrays and the processing described in U.S. Pat. Nos. 5,545,531 and 5,874,219. These patents are incorporated herein by reference in their entireties for all purposes.

Additionally, the present invention may have preferred embodiments that include methods for providing genetic information over networks such as the Internet as shown in U.S. patent application Ser. Nos. 10/063,559, 60/349,546, 60/376,003, 60/394,574, 60/403,381.

Definitions

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Array Plate or a Plate a body having a plurality of arrays in which each array is separated from the other arrays by a physical barrier resistant to the passage of liquids and forming an area or space, referred to as a well.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs) as described in U.S. Pat. No. 6,156,501 that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementary exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Excitation energy refers to energy used to energize a detectable label for detection, for example illuminating a fluorescent label. Devices for this use include coherent light or non coherent light, such as lasers, UV light, light emitting diodes, an incandescent light source, or any other light or other electromagnetic source of energy having a wavelength in the excitation band of an excitable label, or capable of providing detectable transmitted, reflective, or diffused radiation.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5×SSPE) and a temperature of from about 25° C. to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning: A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Isolated nucleic acid is an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

Label for example, a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Microtiter plates are arrays of discrete wells that come in standard formats (96, 384 and 1536 wells) which are used for examination of the physical, chemical or biological characteristics of a quantity of samples in parallel.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 20, 25, 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Reader or plate reader is a device which is used to identify hybridization events on an array, such as the hybridization between a nucleic acid probe on the array and a fluorescently labeled target. Readers are known in the art and are commercially available through Affymetrix, Santa Clara Calif. and other companies. Generally, they involve the use of an excitation energy (such as a laser) to illuminate a fluorescently labeled target nucleic acid that has hybridized to the probe. Then, the reemitted radiation (at a different wavelength than the excitation energy) is detected using devices such as a CCD, PMT, photodiode, or similar devices to register the collected emissions. See U.S. Pat. No. 6,225,625.

Receptor: A molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

WGSA (Whole Genome Sampling Assay) Genotyping Technology: A technology that allows the genotyping of thousands of SNPs simultaneously in complex DNA without the use of locus-specific primers. In this technique, genomic DNA, for example, is digested with a restriction enzyme of interest and adaptors are ligated to the digested fragments. A single primer corresponding to the adaptor sequence is used to amplify fragments of a desired size, for example, 500-2000 bp. The processed target is then hybridized to nucleic acid arrays comprising SNP-containing fragments/probes. WGSA is disclosed in, for example, U.S. Provisional Application Ser. Nos. 60/319,685, 60/453,930, 60/454,090 and 60/456,206, 60/470,475, U.S. patent application Ser. Nos. 09/766,212, 10/316,517, 10/316,629, 10/463,991, 10/321,741, 10/442,021 and 10/264,945, each of which is hereby incorporated by reference in its entirety for all purposes.

Reference will now be made in detail to exemplary embodiments of the invention. While the invention will be described in conjunction with the exemplary embodiments, it will be understood that they are not intended to limit the invention to these embodiments. On the contrary, the invention is intended to cover alternatives, modifications and equivalents, which may be included within the spirit and scope of the invention.

II. Patterned Excitation Detection of Targets with Periodic Regular Geometric Configuration

In one aspect of the invention, methods and devices are provided for microarray detection using a series of structured, textured, or patterned excitation (referred herein as patterned excitation) images to achieve subpixel resolution in detecting probe intensities. As used herein, subpixel refers to an area of a target region that is smaller than that of a single pixel in a detector. For example, in a 6.4 μm CCD detector. The size of one pixel of the CCD detector is about 6.4 μm×6.4 μm. Subpixel detection means detecting intensities of probes whose feature size is smaller than that the dimension of the detector pixel.

In such detections, probe feature information (the periodic geometry configuration of probes on a microarrary) is typically incorporated into the analysis. In some embodiments, electron multiplying CCDs are used for imaging fluorescence emission patterns which indicate hybridization between probes and targets. However, as one of skill in the art would appreciate, this invention is not limited to any particular detection devices. Photo detection arrays (e.g., CCD, APS) are generally preferred. However, single detector, such as a PMT tube, could also be used.

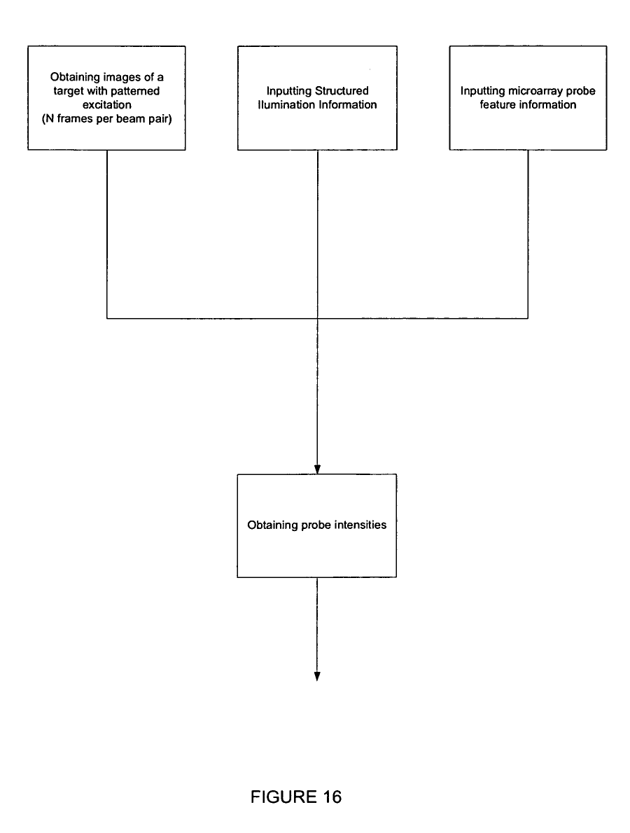

The typical components for the patterned excitation detection system of the invention include pattern excitation imaging device and a computer system for analyzing the images with structured illumination information and microarray probe feature information (see, e.g., FIG. 16).

A. Patterned Excitation

Some of basic theories and practical devices for generating interference patterns on an object to enhance imaging resolution of various objects are described in, for example, U.S. Provisional Application No. 60/559,806, filed on Apr. 6, 2004; and U.S. Provisional Application No. 60/565,041, filed on Apr. 23, 2004; and U.S. patent application Ser. No. 10/026,615; Jekwan, Ryu, Resolution Improvement in Optical Microscopy by Use of Multibeam Inteferometric Illumination, September 2003, MIT Ph.D., Dissertation, incorporated herein by reference (including all the references cited in the dissertation); J. W. Goodman, Introduction to Fourier Optics, McGraw-Hill, Boston, 1996; B. Bailey, D. L. Farkas, D. L. Taylor, F. Lanni, Nature 366, 44 (1993); M. A. A. Neil, R. Juŝkitis, T. Wilson, Opt. Lett 22, 1905 (1997); R. Heintzmann, C. Cremer, Proc. SPIE 3568, 185 (1998); M. G. L. Gustafsson, D. A. Agard, J. W. Sedat, J. Microsc. 195, 10 (1999); M. G. L. Gustafsson, J. Microsc. 198, 82 (2000); J. T. Frohn, H. F. Knapp, A. Stemmer, Proc. Natl. Acad. Sci. U.S.A. 97, 7232 (2000); V. Krishnamurthi, B. Bailey, F. Lanni, Proc. SPIE 2655, 18 (1996); G. E. Cragg, P. T. C. So, Opt. Lett 25, 46 (2000); J. T. Frohn, H. F. Knapp, A. Stemmer, Opt. Lett 26, 828 (2001); P. T. C. So, H. S. Kwon, C. Y. Dong, J. Opt. Soc. Am. A 18, 2833 (2001); M. S. Mermelstein, PhD Thesis, Massachusetts Institute of Technology (2000); M. Born, E. Wolf, Principles of Optics (Cambridge University Press, Cambridge, 1980), all incorporated herein by reference.

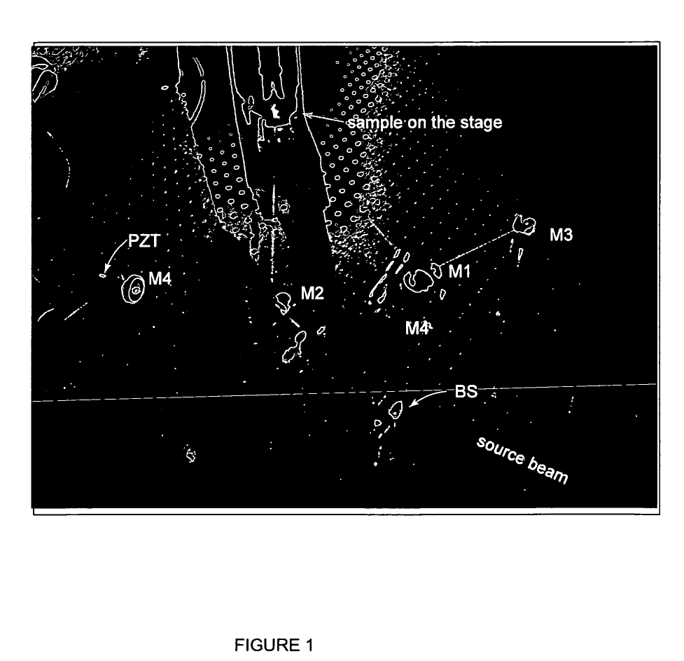

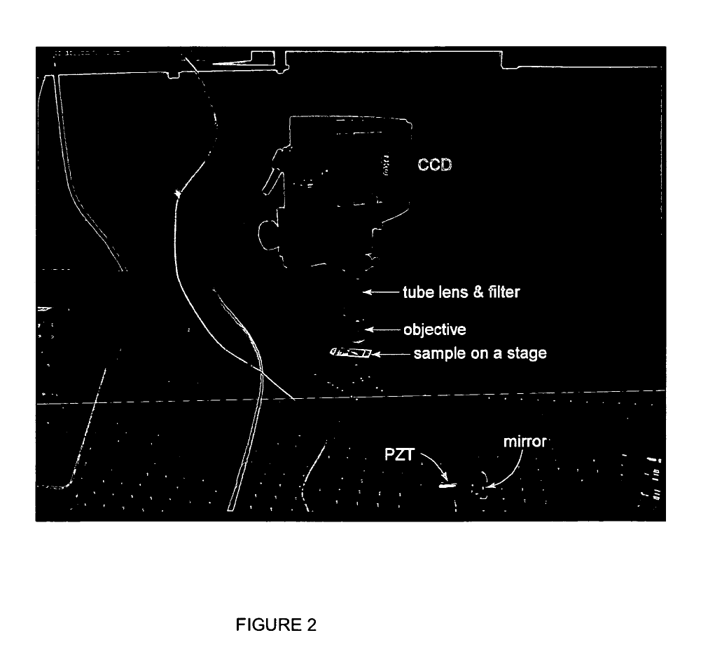

FIG. 1 shows an exemplary Experimental setup that generates patterned excitation using the interference of two coherent beams. A single source beam (model 177-G11-FBR, Spectra-Physics, Mountain View, Calif.) with 488 nm wavelength was split into two beams using a non-polarizing beam splitter (BS). The source beam was a linearly polarized with 0.7 mm beam diameter. Each of the two beams from the beam splitter was steered by two mirrors (M1-M3 or M2-M4) until they overlap and produce patterned excitation onto the sample region on the stage. Fluoresced photons from the sample are collected and recorded using a CCD imaging setup described in FIG. 2. The beams travel horizontally relative to the table until they reflected off the two final mirrors; M1 and M2. These two mirrors are tilted such that the beams travel downward after the reflection. For both beams, the angle between a line perpendicular to the horizontal surface and the beam was roughly 75 degrees. One of the steering mirrors, M4, was glued to a piezo-electric transducer (PZT, model P-841.10, Physik Instrument, Tustin, Calif.). By controlling the position of the mirror using PZT, the optical phase of one of the two beams can be controlled. Upon excited by the patterned excitation described in FIG. 1, a sample emits fluorescent photons that are collected and recorded by a CCD imaging setup shown in this photograph (FIG. 2). A standard Affymetrix cartridge was used as a sample is shown on top of a stage. The fluoresced photons from the sample are collected by a microscope objective (model CFI Plan Fluor 10×, Nikon Instruments Inc., Melville, N.Y.) and then pass through a long-pass filter (model CG-OG-515-1.00-3, CVI Laser Corporation, Livermore, Calif.) and a tube lens (model NT56-125, Edmund Industrial Optics, Barrington, N.J.) before they are projected onto a CCD camera (model DV887-FI, Andor Technology, South Windsor, Conn.).

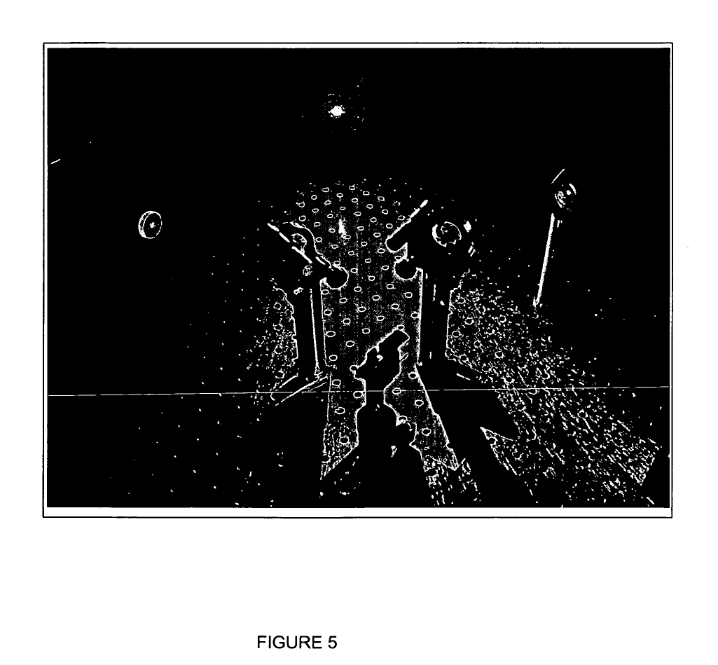

FIG. 5 show the direct imaging of the interference pattern that can be generated using the device in FIGS. 1 and 2. To directly verify the generation of the high resolution optical pattern formed by the interference of two laser beams, a high power microscope objective with 100× magnification (model NT38-344, Edmund Industrial Optics, Barrington, N.J.) was used. The objective was positioned such that the focal plane of the objective lies in the region where the two beams overlap (bright spot in the photograph).

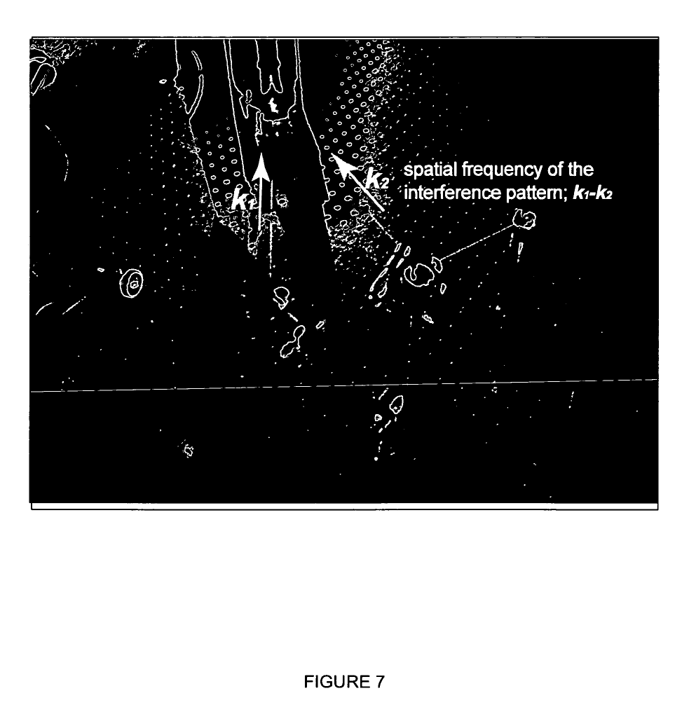

FIG. 7 shows the directions of the beams determines the spatial frequency of the projected interference pattern. The figure above shows the propagation vectors, or direction vectors, of the two interfering beams, indicated as vectors k₁ and k₂, respectively. The spatial frequency of resulting interference pattern is equivalent to the difference between these two vectors, that is, k₁−k₂. Directions of the two interfering beams can be conveniently defined in terms of angle. The separation angle φ on the figure left defines an angle between the two laser beams looking from the top. The half cone angle θ on the right figure corresponds to an angle between the laser beam and a line perpendicular to the horizontal surface. The angle θ is identical for both beams.

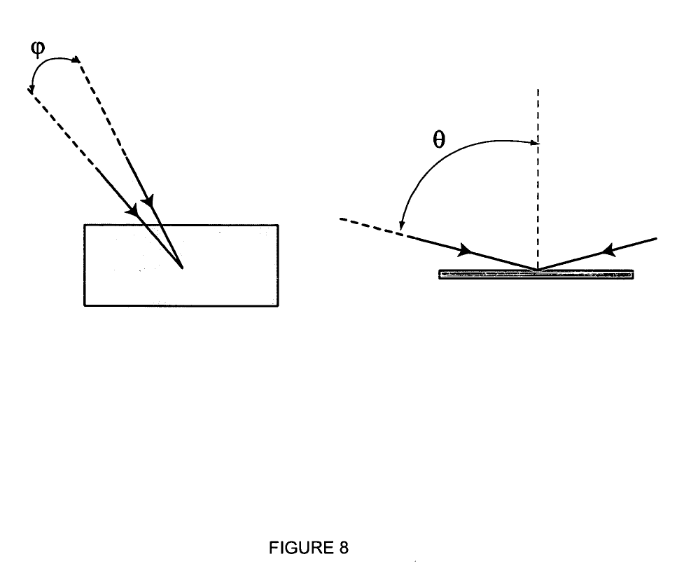

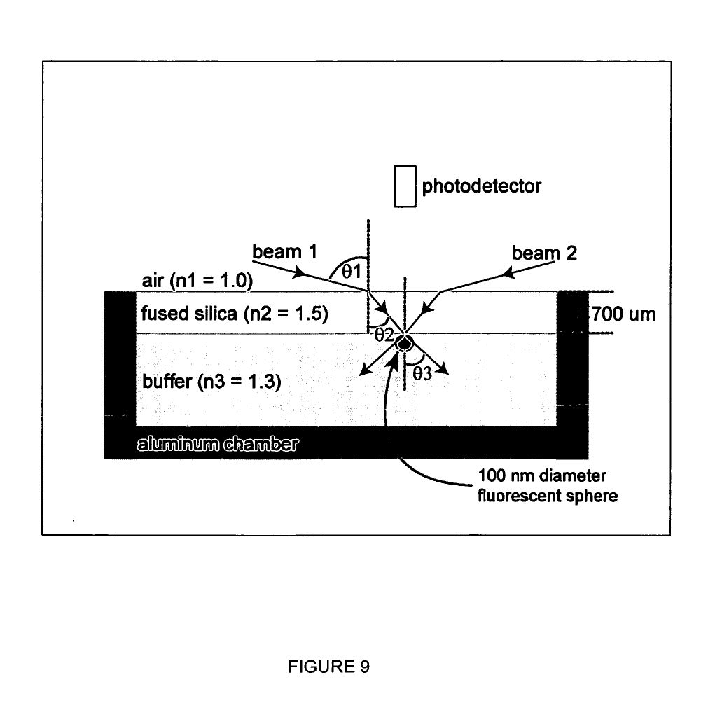

FIG. 9 show generating patterned excitation in the probe region of Affymetrix microarray. Unlike the situation where the two beams interfere in free air space, the beams undergo multiple refractions (and reflections as well) as they pass through several heterogeneous regions. In case of the standard Affymetrix microarray, the beam from the air with refractive index n₁=1.0 pass through the 700 um thick fused silica layer with refractive index n₂=1.5 (for 488 nm wavelength), and finally reaches the probe region with refractive index n₃=1.3. The half cone angles defined in FIG. 8 at each different layer are indicated in the figure. When the cone angle in the air (θ₁) is 75 degrees, the resulting cone angle in the probe region (θ₃) becomes 48 degrees. The figure also shows a 100 nm diameter fluorescent sphere bonded to the bottom surface of the fused silica substrate. The size of this sphere was chosen to be a fraction of the wavelength of light such that the particle can spatially sample the resulting interference pattern. When the interference pattern is translated relative to the sphere, the brightness of the sphere will change depending on the position of the interference pattern. A photo detector that is placed on top can detect and record such brightness change.

In one aspect of the invention, a highly sensitive and high speed imaging device, such as an electron multiplying CCD (EM CCD), is used to detect the emission pattern of a hybridized microarray. The microarray can be a nucleic acid probe array such as a spotted array (e.g., with cDNA or short oligonucleotide probes), high density in situ synthesized arrays (such as the GeneChip® high density probe arrays manufactured by Affymetrix, Inc., Santa Clara, Calif.). The microarrays can also be protein or peptide arrays. Typically, the density of the microarrays is higher than 500, 5000, 50000, or 500,000 different probes per cm². The feature size of the probes is typically smaller than 500, 150, 25, 9, or 1 μm². The locations of the probes can be determined or decipherable. For example, in some arrays, the specific locations of the probes are known before binding assays. In some other arrays, the specific locations of the probes are unknown until after the assays. The probes can be immobilized on a substrate, optionally, via a linker, beads, etc.

An EMCCD device is used for imaging the fluorescence emission pattern, which is used for biological analysis. EM CCD is a device that unites the sensitivity of Intensified CCD (ICCD) or an electron bombardment CCD (EBCCD), while retaining the inherent benefits of a CCD. For a description of the EMCCD technology, see, e.g., EP 08 866 501, incorporated herein by reference. The application of EMCCD enables fast detection of weak signals. For example, for detecting hybridization patterns in nucleic acid probe arrays, the exposure time can be shorter than 1000, 800, 600, 500, 400, 300, 200, 100, 80, 60, 40, 20, or msec.

B. Microarray Analysis Using Patterned Excitation Images

In one aspect of the invention, a series of images (frames) of a microarray, such as a nucleic acid array that has been hybridized with a target that is labeled with a fluorescent label, are obtained using patterned excitation. Such images are then processed based upon the knowledge the excitation patterns employed and the probe feature information, such as probe spacing, set backs, feature size, presumed dynamic range, etc. (FIG. 16).

Microarrays (including bead arrays) typically have periodic repetition of probes that are synthesized or otherwise immobilized on to a substrate. The probe features typically assume somewhat regular geometric shape such as square, rectangular or circular. For example, GeneChip® high density oligonucleotide probe arrays have square features with set backs (separation between intended synthesis areas). The information about the periodic repetition of probes is used to facilitate the extraction of probe intensities from the series of images obtained using patterned excitation.

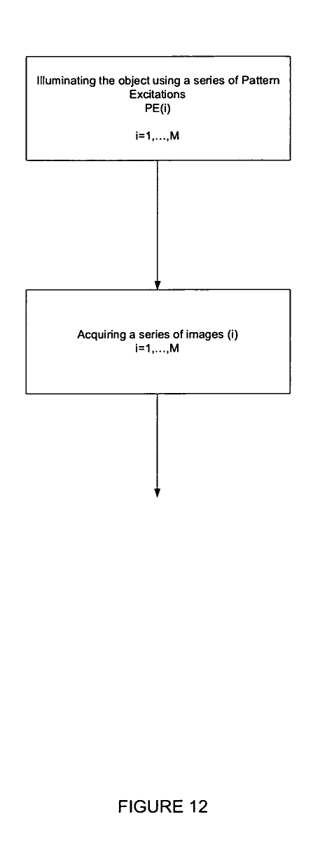

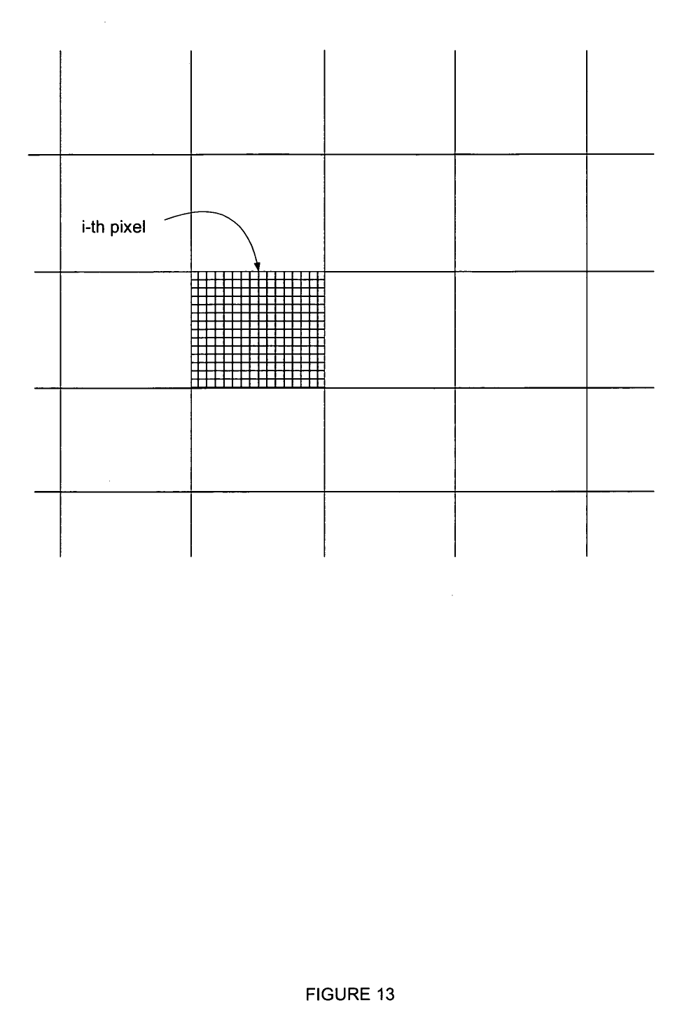

FIG. 12 shows a high level view of one exemplary way of organizing imaging data. FIG. 13 shows subpixels with a detector pixel. The large squares represent the physical pixel of a detector. The small square within a large square represents subpixels whose values are going to be determined through post processing. The raw images only contain intensity values for the pixels, not the subpixels.

Some mathematical symbols can be conveniently used to illustrate algorithms that are useful for analysis. One of skill in the art would appreciate that mathematical representations used herein are for illustrating purposes. Alternative representations are well within the scope of the invention. For this specification, i is the pixel number index, i=1, . . . , L; Iⁱ(m,n) is subpixel intensity of i-th pixel at a subpixel location (m,n); Wⁱ(m,n, k) is the weighting function within i-th pixel for k-th frame at a subpixel location (m,n), and finally, bⁱ(k) is the sequence of gray intensity values of i-th pixel.

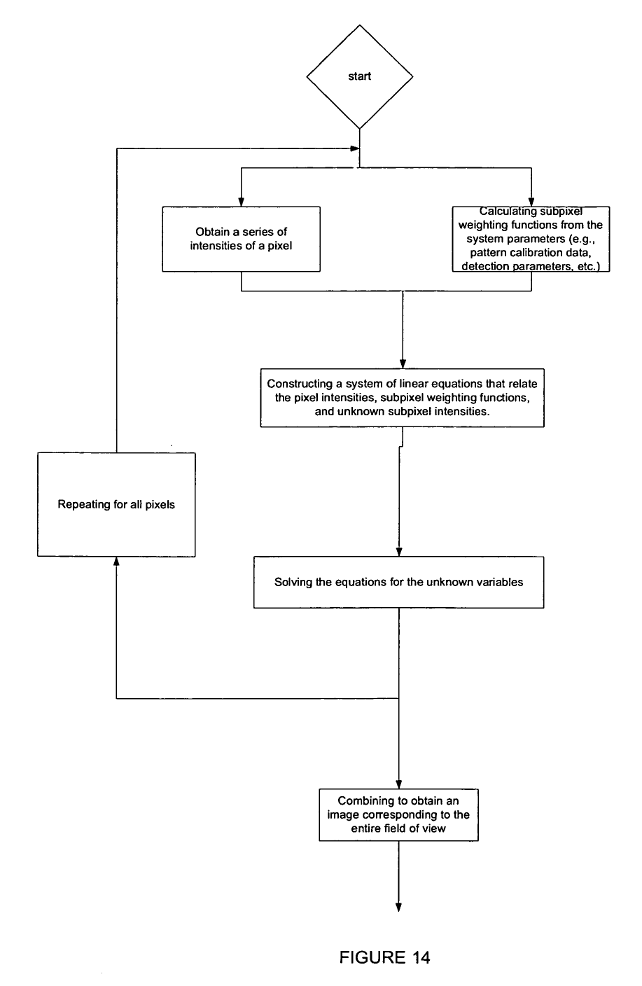

FIG. 14 shows one exemplary embodiment of the algorithm for constructing an image. The series of intensities may be represented by bⁱ(k); weighting functions would be Wⁱ(m,n, k); and the equations can be

$${b}^{i} ( k ) = {\sum }_{m} {\sum }_{n} {W}^{i} ( m . , n , k ) {I}^{i} ( m , n ) ,$$

where Iⁱ(m,n) is the unknown subpixel intensities.

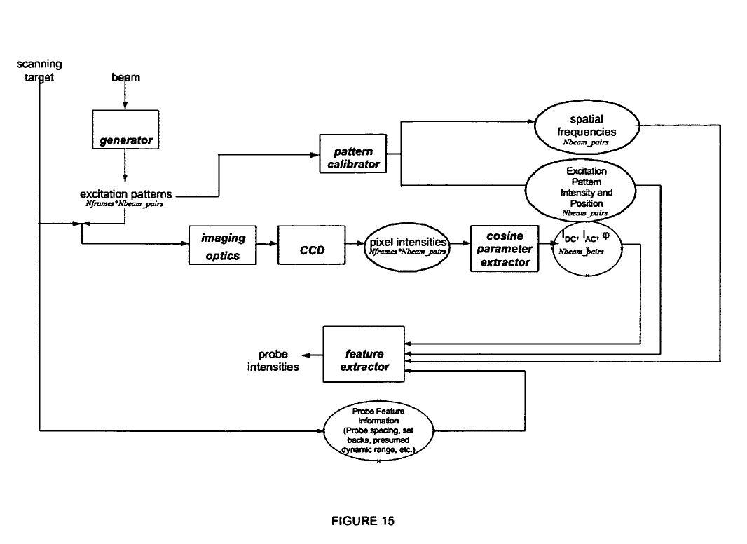

The bⁱ(k) may be subject to cosine parameter extractor to obtain cosine parameters (such as I_{DC}, I_{AC} and φ) to generate a reconstructed sinusoidal function the samples of which can be used instead of the original bⁱ(k) To compensate for the noise in measuring pixel intensities (see, FIG. 15).

The weighting function can be determined from the system parameters such as the excitation pattern spatial frequencies, positions, intensities as well as detector parameters such as NA (numerical aperture) of the imaging lens and the pixel response function. These are determined from the pattern calibration, also can be measured independently in addition to manufacture specifications.

In some embodiments, it is not necessary to obtain the parameters as long as the parameters are the same for a calibration process and a detection process. During calibration, multiple reference samples with known subpixel intensities may be used to obtain a series of images. The equation

$${b}^{i} ( k ) = {\sum }_{m} {\sum }_{n} {W}^{i} ( m . , n , k ) {I}^{i} ( m , n )$$

can be solved for Wⁱ(m,n, k), because bⁱ(k) is detectable and Iⁱ(m,n) is known. With m×n number of reference samples, the equation can be easily solved. However, because Wⁱ(m,n, k) reflects structured illumination data, a reduced set of equation with reduced number of reference samples may be sufficient in some cases. For a review of linear Algebra, see, e.g., “Linear Algebra and Its Applications (3rd Edition)” by David C. Lay, Addison Wesley; 3 edition (Jul. 18, 2002), ISBN: 0201709708, incorporated herein by reference.

In some other embodiments, the Wⁱ(m,n, k) is an excitation light intensity distribution or profile. This distribution can be calculated, for example, using pattern calibration parameters as follows: E_{DC}+E_{AC}·cos(kₓ·x+k_{y}·y+φ), where E_{DC} and E_{AC} are DC and AC components of the pattern intensities, respectively; kₓ and k_{y} are x and y components of the pattern spatial frequency, respectfully; and finally the φ represents subpixel position of the pattern reference to, for example, the center of the pixel. For a review of light interference theory, see, e.g., “Introduction To Fourier Optics” by Joseph W Goodman, McGraw-Hill Science/Engineering/Math; 2 edition (Jan. 1, 1996) ISBN: 0070242542, incorporated herein by reference.

In some embodiments, the Wⁱ(m,n, k) may be stable for a system for a period of time and no new calculation is necessary during this period to generate subpixel intensity values.

In some other embodiments, the calibration and determination of Wⁱ(m,n, k) may be conducted every time a sample is imaged, where border probes or other control probes may be used for dynamic calibration purposes. In some cases, Wⁱ(m,n, k) may be calculated for control probes as a quality control or diagnostic measure.

Once Wⁱ(m,n, k) is available either by factory supplied data, through calibration or dynamic calibration, one can easily solve the equation

$${b}^{i} ( k ) = {\sum }_{m} {\sum }_{n} {W}^{i} ( m . , n , k ) {I}^{i} ( m , n )$$

to obtain individual subpixel intensity values (FIG. 14). This process can be repeated for each pixel to generate a combined image of an entire field of view.

In another aspect of the invention, the intensity values are estimated using optimization methods. Methods for estimating parameters using optimization methods are well known in the art and are described in, for example, “Optimization Theory With Applications” by Donald Pierre, Dover Publications (Oct. 1, 1986) ISBN: 048665205X; “Handbook of Applied Optimization” by P. M. Pardalos (Editor), Mauricio G. C. Resende (Editor), Panos M. Pardalos (Editor), Oxford University Press (Apr. 1, 2002) ISBN: 0195125940; “Numerical Optimization” by Jorge Nocedal, Stephen J. Wright, Springer; 1 edition (Aug. 27, 1999) ISBN: 0387987932.

In some embodiments, the subpixel intensities are estimated with probe feature information as constraints. For example, the regularity of the probe features is used as constraints. The dynamic range the probe intensities can also be used. One particularly preferred method is to minimize

$${‖ {b}^{i} ( k ) - {\sum }_{m} {\sum }_{n} {W}^{i} ( m . , n , k ) {I}^{i} ( m , n ) ‖}^{2} .$$

Liner programming is a preferred method for estimating the intensity values. Methods and computer software products for performing linear programming are well known in the art. Software products are commercially available. See, also, “Numerical Recipes in C++: The Art of Scientific Computing” by William H. Press (Editor), Saul A. Teukolsky (Editor), William T. Vetterling, Brian P. Flannery, Cambridge University Press; 2 edition (February, 2002) ISBN: 0521750334; “Linear Programming (Series of Books in the Mathematical Sciences)” by Vasek Chvatal, W. H. Freeman (Sep. 15, 1983) ISBN: 0716715872, all incorporated herein by reference.

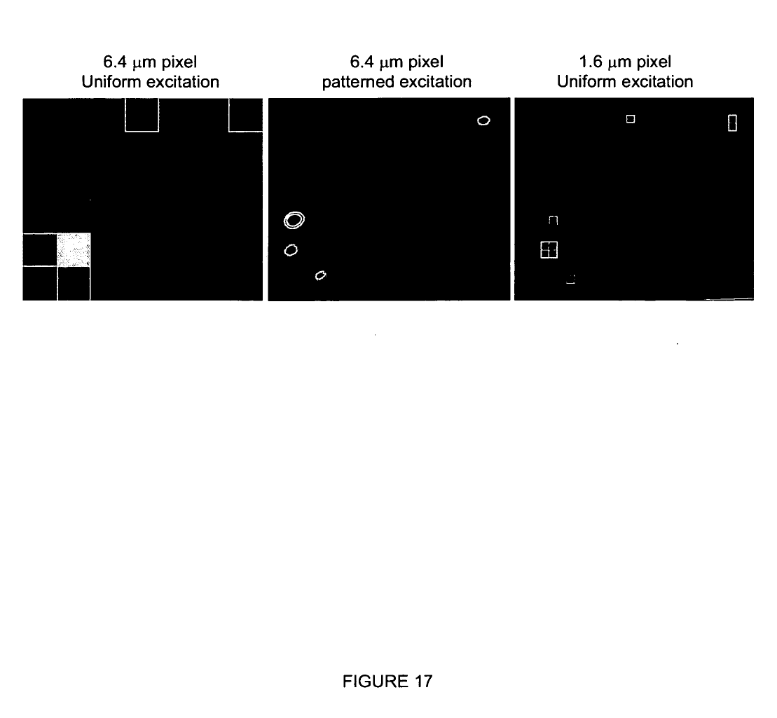

In one example, a hybridized and stained GeneChip® U133 AB plus 5 micron probe array (Affymetrix, Santa Clara, Calif.) was used to demonstrate the method of the invention. FIG. 17 demonstrates spatially resolving subpixel probe intensities with patterned excitation and foruier sum algorithm (described in Jekwan, Ryu, Resolution Improvement in Optical Microscopy by Use of Multibeam Inteferometric Illumination, September 2003, MIT Ph.D., Dissertation, incorporated herein by reference). The figure on the left is a conventional CCD image of a small region of a microarray with 5 μm probe spacing imaged using 6.4 μm image pixel size under conventional CCD imaging conditions; the image at the middle is a computer reconstructed image of the same region of the array acquired using the same lens and CCD with pattern excitation. Comparing the two images demonstrates the improvement in resolution, resolving subpixel intensities with the patterned excitation. The image on the right was acquired using 1.6 μm pixel size under conventional CCD imaging condition, showing a good correspondence with the patterned excitation image acquired at 6.4 μm pixel.

In another aspect of the invention, computer software products are provided for microarray analysis. The software products typically include a computer readable medium with computer codes that execute the methods of the invention.

In yet another aspect of the invention, systems for microarray analysis are provided. Typically, such a system includes a computer with a central processing unit coupled with a memory for executing computer code that performs the methods of the invention. A system of the invention may also include a patterned excitation unit that has a excitation source beam, a pattern generator, image optics, an imager (such as a CCD)(FIG. 15). The computer unit may control the patterned excitation unit and receive the data from the image. The computer unit may contain computer software codes that perform pattern calibration, cosine parameter extraction, feature extraction, etc.

### CONCLUSION

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

1. A computer readable medium comprising computer-executable instructions for performing the method comprising:
Obtaining a series of fluorescent images of a microarray, wherein the fluorescent signals reflect binding between targets and probes, and wherein each of the images is obtained with a different excitation pattern; and
Analyzing said images using calibrated information about said different excitation patterns to obtain intensities for each of said probes.

2. The computer readable medium of claim 1 wherein said analyzing comprises generating a composite image wherein said composite image has a higher resolution than those of said fluorescent images.

3. The computer readable medium of claim 1 wherein obtaining comprises obtaining said images using a photo detection array.

4. The computer readable medium of claim 1 wherein the information about different excitation patterns comprises excitation pattern intensities and positions.

5. The computer readable medium of claim 1 wherein said analyzing comprises extracting cosine parameters to obtain I_{DC}, I_{AC}, and φ of pixel intensities.

6. The computer readable medium of claim 1 wherein the analyzing comprises calculating subpixel weighting functions from system parameters.

7. The computer readable medium of claim 6 wherein the analyzing further comprises constructing a system of linear equations that relate the pixel intensities, subpixel weighting functions, and unknown subpixel intensities.

8. The computer readable medium of claim 7 wherein the linear equations are:
$${b}^{i} ( k ) = {\sum }_{m} {\sum }_{n} {W}^{i} ( m . , n , k ) {I}^{i} ( m , n ) ,$$
wherein Iⁱ(m,n) is the unknown subpixel intensities; Wⁱ(m,n, k) is the weighting function within i-th pixel for k-th frame at a subpixel location (m,n); and bⁱ(k) is the sequence of gray intensity values of i-th pixel.

9. The computer readable medium of claim 8 wherein said analyzing further comprises solving said equations.

10. The computer readable medium of claim 9 wherein said analyzing further comprises combining subpixel intensity information for each pixel to obtain an image corresponding to an entire field of view.

11. The computer readable medium of claim 10 wherein said Wⁱ(m,n, k) can be calculated, for example, using pattern calibration parameters as: E_{DC}+E_{AC}·cos(kₓ·x+k_{y}·y+φ), wherein E_{DC} and E_{AC} are DC and AC components of the pattern intensities, respectively; kₓ and k_{y} are x and y components of the pattern spatial frequency, respectfully; and the φ represents subpixel position of the pattern.

12. The computer readable medium of claim 11 wherein the Wⁱ(m,n, k) is calculated by solving the equation
$${b}^{i} ( k ) = {\sum }_{m} {\sum }_{n} {W}^{i} ( m . , n , k ) {I}^{i} ( m , n )$$
using data obtained with reference samples with known subpixel intensities.

13. A computer readable medium comprising computer-executable instructions for performing the method comprising:
Obtaining a series of fluorescent images of a microarray, wherein the fluorescent signals reflect binding between targets and probes, and wherein each of the images is obtained with a different excitation pattern; and
Analyzing said images using calibrated information about said different excitation patterns and probe feature information to obtain intensities for each of said probes.

14. The computer readable medium of claim 13 wherein said analyzing comprises generating a composite image wherein said composite image has a higher resolution than those of said fluorescent images.

15. The computer readable medium of claim 14 wherein said information about different excitation patterns comprises spatial frequency information for each beam pair.

16. The computer readable medium of claim 15 wherein said spatial frequency information comprises orientation and spacing between adjacent peak intensities of the interference pattern.

17. The computer readable medium of claim 16 wherein the information about different excitation patterns comprises excitation pattern intensities and positions.

18. The computer readable medium of claim 17 wherein said analyzing comprises extracting cosine parameters to obtain I_{DC}, I_{AC}, and φ of pixel intensities.

19. The computer readable medium of claim 18 wherein the analyzing comprises calculating subpixel weighting functions from system parameters.

20. The computer readable medium of claim 19 wherein the analyzing further comprises estimating subpixel intensities using pixel intensities using said probe feature information as constraints using an optimization method.

21. The computer readable medium of claim 20 wherein the linear programming method comprises minimizing
$${‖ {b}^{i} ( k ) - {\sum }_{m} {\sum }_{n} {W}^{i} ( m . , n , k ) {I}^{i} ( m , n ) ‖}^{2} ,$$
wherein Iⁱ(m,n) is the unknown subpixel intensities; Wⁱ(m,n, k) is the weighting function within i-th pixel for k-th frame at a subpixel location (m,n); and bⁱ(k) is the sequence of gray intensity values of i-th pixel.

22. The computer readable medium of claim 21 wherein said minimizing comprises using linear programming with said constraints.

23. The computer readable medium of claim 22 wherein said constraints comprise the regularity of probe features.

24. The computer readable medium of claim 23 where said constraints comprise expected range of the subpixel intensities.

25. The computer readable medium of claim 24 wherein said analyzing further comprises combining subpixel intensity information for each pixel to obtain an image corresponding to an entire field of view.

26. The computer readable medium of claim 25 wherein said Wⁱ(m,n, k) can be calculated, for example, using pattern calibration parameters as: E_{DC}+E_{AC}·cos(kₓ·x+k_{y}·y+φ), wherein E_{DC} and E_{AC} are DC and AC components of the pattern intensities, respectively; kₓ and k_{y} are x and y components of the pattern spatial frequency, respectfully; and the φ represents subpixel position of the pattern.

27. The computer readable medium of claim 26 wherein the Wⁱ(m,n, k) is calculated by solving the equation
$${b}^{i} ( k ) = {\sum }_{m} {\sum }_{n} {W}^{i} ( m . , n , k ) {I}^{i} ( m , n )$$
using data obtained with reference samples with known subpixel intensities.
